(19) United States
(12) Patent Application Publication
(10) Pub. No.: US 2005/0203315 A1
(43) Pub. Date: Sep. 15, 2005

(54) Process for converting alcohols to carbonyl compounds

(76) Inventors: Hansjorg Grutzmacher, Wettswil (CH); Hartmut Schonberg, Kilchberg (CH); Souad Boulmaaz, Birsfelden (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/509,779

(22) PCT Filed: Mar. 26, 2003
(86) PCT No.: PCT/EP03/50079

§ 371 Date: Sep. 29, 2004

(30) Foreign Application Priority Data
Apr. 5, 2002 (EP) 024005269.8

(51) Int. Cl. C07C 45/36; C07F 1/08

(52) U.S. Cl. 568/360; 546/2

(57) ABSTRACT

The present invention provides a process for selectively converting dihydroxy-or polyhydroxy alcohols into carbonyl compounds which process comprises the step of oxidizing the dihydroxy-or polyhydroxy alcohols with dioxygen (O₂) as oxidant in the presence of a catalytic system comprising (1) a copper salt, a copper salt containing a heterocyclic ligand or a copper complex salt, and (2) a base.

The invention relates to a catalytic oxidative process for selectively converting polyhydroxy alcohols into the corresponding carbonyl compounds.

The oxidation of primary or secondary alcohols to carbonyl compounds in the presence of a catalytic system comprising (1) a catalytic amount of a copper (I) salt; (2) a bidentate ligand such as, for example, a phenanthroline ligand and (3) a base such as, for example, a carbonate has been described in U.S. Pat. No. 5,912,388. This Patent-Publication does not give any hint how to oxidize polyhydroxy alcohols.

It is known that copper complexes when used in aerobic oxidation reaction of diols cause C—C bond cleavage reactions and/or lead to over-oxidation giving carbonic acids as final products. (L. Prati, M- Rossi, J. Mol. Cat., A: Chem., 1996, 110, 221; or K. K. Sen Gupta, B. N. Nandy, S. Sen Gupta, J. Chem. Res. 1993, 396.)

It has now been found that C—C bond cleavage can be avoided by using a catalytic system as defined below.

The present invention provides a process for selectively converting dihydroxy- or poly-hydroxy alcohols into carbonyl compounds using dioxygen (O₂) as oxidant in the presence of a catalytic system comprising
(1) a copper salt, a copper salt containing a heterocyclic ligand or a copper complex salt, and
(2) a base.

Dihydroxy and polyhydroxy alcohols are suitable for the purpose of this invention. Examples of dihydroxy alcohols include 1,2-diols such as for example ethylene glycol, propane-1,2-diol, butane-1,2-diol; diols such as for example 1,3-propane diol, 1,4-butane diol, longer chain α,β-C₅-C₁₈alkyldiols or α,ω-C₅-C₁₈alkyldiols such as, for example, pentane diols, hexane diols, octane diols, dodecane diol, diethylene glykol, triethylenglykol, cyclic diols such as 1,3-cyclopentane diol, 1,2-, 1,3- or 1,4-cyclohexane diol, and the like. Examples of polyhydroxy alcohols include glycerol, pentaerythritol, sorbitol, sugar, starch, and the like.

The copper salt is selected from CuCl, CuBr, Cul, CuNO₃, CuBF₄, CuSO₄, CuPF₆ and the like. CuCl is preferred.

Suitable ligands are preferably bidendate ligands such as for example 1,10-phenanthroline or substituted phenanthrolines such as, for example, 5-methyl-1,10-phenanthroline, 2,9-di-methyl-1,10-phenanthroline, 4,7-dimethyl-1,10-phenanthroline, 3,4,7,8-tetramethyl-1,10-phenanthroline, 4,7-dihydroxy-1,10-phenanthroline, bathophenantroline, bathocuproinedi-sulfonate, 2,2′-bipyridine, 2,2′-bipyridyl-3,3′-dicarboxylate, 2,2′-biquinoline, bis(2-pyridylethyl)-amine, tris(2-pyridylethyl-)amine, 2-pyridyl-(N-tert.butyl)-methylimine, (2-pyridyl)methanol, ethylene(2,5-dihydroxy-phenylimine) or bis(2-hydroxy-3,5-di(tert.butyl)phenyl)sulfide. Phenanthroline ligands are preferred.

The copper complex salt is preferably an alkali metal salt [M₄(Cu₄OCl₁₀)] or [M(CuCl₃)] or [M₂(CuCl₄)] or mixtures thereof wherein M is an alkali metal cation; an ammonium salt [R₁R₂R₃R₄N]₄(Cu₄OCl₁₀)] or [R₁R₂R₃R₄N](CuCl₃)] or [R₁R₂R₃R₄N]₂(CuCl₄)] or mixtures thereof wherein R₁-R₄ is independently of one another C₁-C₆alkyl, phenyl or benzyl. Examples of complexes wherein R₁-R₄ are identical are [(CH₃)₄N]₄(Cu₄OCl₁₀)] or [(C₂H₅)₄N]₄(Cu₄OCl₁₀)] and the like. Suitable are also complexes wherein R₁-R₄ are not identical such as, for example, benzyl triethyl and benzyl trimethyl ammonium copper complexes or phenyl triethyl and phenyl trimethyl ammonium copper complexes.

The preparation of the copper complexes is known and for example described by J. A. Bertrand and J. A. Kelley, in Inorganic Chemistry, 8 (9), (1969) 1982.

The copper salt and the ligand form a complex such as for example Cu₂(μ₂—Br)bipy₂ derived from the reaction of 2,2′-bipyridine and CuBr; or [Cu(phen)₂]⁺ CuCl₂⁻ derived from the reaction of a phenanthroline, for example 1,10-phenanthrolin and CuCl.

The molar ratio of the alcohol to copper salt is in the range from 1: (0.01-0,1).

The base may be a hydroxide, an oxide or a carbonate such as, for example, Li(OH); NaHCO₃; Na₂CO₃; Na(OH); K₂CO₃; K(OH); MgO; CaCO₃; Ca(OH)₂; BaCO₃; Al₂O₃ (basic); a quaternary ammonium salt or a hydrate thereof such as, for example, [R₁R₂R₃R₄N](OH); [R₁R₂R₃R₄N](Hal), wherein Hal is halogen and R₁-R₄ is as defined above; an alcoholate such as, for example, Na(OR₅), K(OR₅) wherein R₅ is C₁-C₆ alkyl, for example methyl, ethyl or tert.butyl; a heterogeneous basic supports selected from amberlite, ambersep, sepiolite, hydrotalcit or bentonit.

Alkyl groups may be linear or branched. Hal is fluorine, chlorine, bromine or iodine, preferably chlorine.

The amount of the base depends on the base used. Using a quaternary ammonium salt as base the mole ratio of copper salt to base is, for example, about 1:2.

In one embodiment of the invention a quaternary ammonium salt or a hydrate thereof, preferably tetramethylammonium hydroxide is used as base. Said system shows a much higher catalytic activity as systems using e.g. K₂CO₃. Furthermore, the amount of the base can be lowered.

The process of the present invention is preferably carried out in the presence of a solvent. Suitable solvents include aromatic solvents (such as benzene, toluene, p-xylene, fluoro-benzene, perfluorobenzene, iso-butyl benzene or mesitylene), nitriles (such as acetonitrile), hydrocarbon solvents (such as petroleum fractions), halogenated solvents (such as dichloro-methane, tetrachloroethylene or 1,2-dichloroethane) or esters (such as methyl or ethyl acetate). Preferred solvents are toluene and acetonitrile.

It is preferred that the process of the invention is carried out at elevated temperature, such as in the range 30-140° C., particularly 60-110° C., preferably 70-90° C.

The process of the present invention can be carried out at atmospheric pressure or at elevated pressure. Preferred is atmospheric pressure.

Oxygen can be supplied in pure form or in the form of air. A gentle stream of oxygen or air was passed over the heterogeneous reaction mixture.

The reaction time varies between 30 min and 2 h depending, for example, on the base used.

The catalyst can be easily recycled by filtration and reactivation with a quaternary ammonium hydroxide or a potassium alcoholate.

When necessary a reductant such as for example selected from the group consisting of Zn, hydrazines, SO₂, Na₂S₂O₄ may be added.

The following Examples explain the invention in more detail,

### EXAMPLE 1

### Catalytic aerobic oxidation of 1-(hydroxy-phenylmethyl) cyclohexanol, using CuCl/phen/K₂CO₃

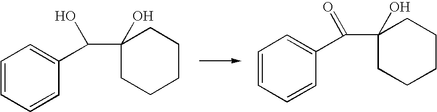

In a 100 ml two-neck, round-bottomed flask equipped with oxygen inlet and reflux condenser were suspended CuCl (22.5 mg, 0.24 mmol) and 1,10-phenanthroline (43.6 mg, 0.24 mmol) in 20 ml of toluene. The mixture was stirred for 10 to 20 min at room temperature. Then K₂CO₃ (1.33 g, 9.68 mmol) and the diol (4.84 mmol) were added successively. The mixture was heated for 30 min in an oil bath at a temperature from about 70° C. to about 90° C. while O₂ was gently passed over the stirred reaction mixture. After filtration the conversion was determined by gas chromatography coupled with mass spectrometry (GC/MS). About 90% conversion is reached within one hour. Alternatively, acetonitrile may be used as solvent. The resulting turn over frequencies (TOF) are about 20 h−1.

### EXAMPLE 2

### Catalytic Aerobic Oxidation of 1-(hydroxy-phenylmethyl) Cyclohexanol Using CuCl/phen/Me₄N(OH).5H₂O:

CuCl (22.5 mg, 0.24 mmol), 1,10-phenanthroline (43.6 mg, 0.24 mmol) and 20 ml of dry toluene (alternatively acetonitrile may be used) were placed in a 100 ml two-necked flask equipped with a gas inlet and reflux condenser. The mixture was stirred for 10 to 20 min at room temperature. Subsequently, Me₄N(OH).5H₂O (87.7 mg, 0.48 mmol) was added. The heterogeneous reaction mixture was stirred for 5 min and then the diol (4.84 mmol) was added. The mixture was heated in an oil bath to about 70° C. for 30 min whereby a gently stream of O₂ was passed over the reaction mixture. After filtration the conversion was determined by gas chromatography (GC/MS).

About 90% conversion is reached within half an hour (TOF=40 h−1).

As seen above the acitivity of the catalytic system can be increased when tetramethyl-ammonium hydroxide pentahydrate is used instead of K₂CO₃. Not only the amount of base can be lowered to about 10 mol % (instead of 200% using K₂CO₃) but also the reaction times become shorter (30 min instead of 60 min); the resulting turn over frequencies (TOF) are about 40 h−1.

### EXAMPLE 3

### Catalytic aerobic oxidation of 1-(hydroxy-phenylmethyl) cyclohexanol using CuCl/Me₄N(OH).5H₂O

CuCl (22.5 mg, 0.24 mmol) were placed with 2 eq. 0.48 mmol [Me₄N(OH).5H₂O] in a two-necked 100 ml flask with gas inlet and reflux condenser and suspended in 20 ml toluene. This off-white slurry became brownish-red when an alcohol was added. The reaction mixture was heated to about 70° C. for 30 min under a gentle stream of O₂. After cooling to room temperature, the crude reaction mixture was analysed by GC/MS. More than 90% conversion to hydroxyketone was found using 1-(hydroxy-phenylmethyl) cyclohexanol as substrate (no cleavage products).

### EXAMPLE 4

### Catalytic aerobic oxidation of 1-(hydroxy-phenylmethyl) cyclohexanol using Me₄N(Cu₄OCl₁₀)/Me₄N(OH).5H₂O as catalyst

The tetra methyl(ammonium) [hexa-μ₂-chloro-μ₄-oxo-tetrachlorocuprate(II)] (100 mg, 0.1 mmol) was suspended in 10 ml of acetonitrile. A trace of Zn powder was added and the mixture stirred for about 5 min. Then Me₄N(OH).5H₂O (200 mg, 1 mmol) and 2.5 mmol of alcohol were added. The mixture was heated for 30 min to about 80° C. whereby the solid became deep brown and the solution shows a reddish colour. The crude reaction mixture was analysed by GC. 70% conversion to hydroxy ketone was observed (no cleavage).

### EXAMPLE 5

The substrates listed in the Table below were oxidised under aerobic conditions using one of the catalytic systems described in Examples 1 and 2

| Educt | Product | Yield % | time Ex. 1 | time Ex. 2 |
|---|---|---|---|---|
| 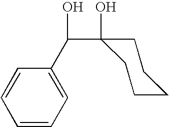 | 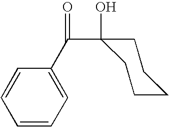 | 94 | 60 | 30 |
| 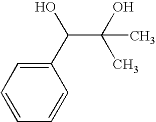 | 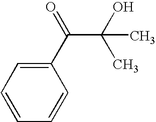 | 97 | 60 | 30 |
| 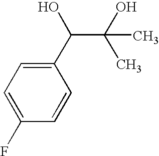 | 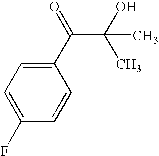 | 87 | 60 | 30 |
| 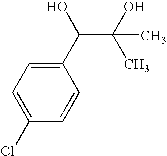 | 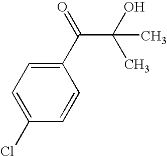 | 99 | 60 | 30 |
| 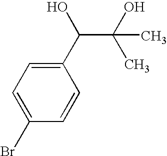 | 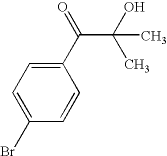 | 95 | 60 | 30 |
| 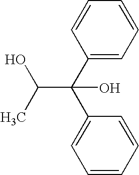 | 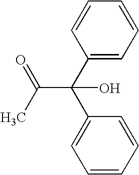 | 69 | 120 | 60 |
| 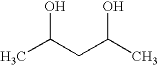 | 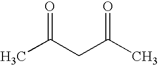 | 60 | 120 | 60 |
| 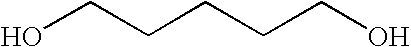 | 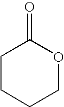 | 30 | 120 | 60 |

### EXAMPLE 6

### Preparation of [Cu(phen)₂]⁺ (CuCl₂)⁻

CuCl was reacted with one equivalent of 1-10-phenanthroline. A deep violet powder forms, which is almost insoluble in all organic solvents. From an acetonitrile solution, single crystals suitable for an X-rayanalysis were obtained. The structure of CuCl/phen consists of the ion pair [Cu(phen)₂]⁺ (CuCl₂)⁻ which was not described in this form in the literature before. In the RAMAN spectrum the Cu—Cl stretching vibration was observed at 308.6 cm⁻¹. Since [Cu(phen)₂]⁺ (CuCl₂)⁻ is a stable compound, this complex was preferably used in aerobic oxidation experiments.

1. A process for selectively converting dihydroxy-or polyhydroxy alcohols into carbonyl compounds which process comprises the step of oxidizing said dihydroxy-or polyhydroxy alcohols with dioxygen (O₂) as oxidant in the presence of a catalytic system comprising
(1) a copper salt, a copper salt containing a heterocyclic ligand or a copper complex salt, and
(2) a base.

2. A process according to claim 1, wherein
the copper salt is selected from the group consisting of CuCl, CuBr, Cul, CuNO₃, CuBF₄, CuSO₄, and CuPF₆;
the ligand is selected from the group consisting of 1,10-phenanthroline, 5-methyl-1,10-phen-anthroline, 2,9-dimethyl-1,10-phenanthroline, 4,7-dimethyl-1,10-phenanthroline, 3,4,7,8-tetramethyl-1,10-phenanthroline, 4,7-dihydroxy-1,10-phenanthroline, batho-phenantroline, bathocuproinedisulfonate, 2,2′-bipyridine, 2,2′-bipyridyl-3,3′-di-carboxylate, 2,2′-biquinoline, bis(2-pyridylethyl)amine, tris(2-pyridylethyl)amine, 2-pyridyl-(N-tert.butyl)-methylimine, (2-pyridyl)methanol, ethylene(2,5-dihydroxy-phenylimine) and bis(2-hydroxy-3,5-di(tert.butyl)phenyl)sulfide;
the copper complex salt is [M₄(Cu₄OCl₁₀)] or [M(CuCl₃)] or [M₂(CuCl₄)] or mixtures thereof wherein M is an alkali metal cation, [R₁R₂R₃R₄N]₄(Cu₄OCl₁₀)] or [R₁R₂R₃R₄N](CuCl₃)] or [R₁R₂R₃R₄N]₂(CuCl₄)] or mixtures thereof wherein R₁-R₄ is independently of one another C₁-C₆ alkyl, phenyl or benzyl; and
the base is selected from the group consisting of Li(OH); NaHCO₃; Na₂CO₃; Na(OH); K₂CO₃; K(OH); MgO; CaCO₃; Ca(OH)₂; BaCO₃; Al₂O₃ (basic); a quaternary ammonium salt or a hydrate thereof [R₁R₂R₃R₄N](OH); [R₁-R₂R₃R₄N](Hal), wherein Hal is halogen and R₁-R₄ is as defined above; an alcoholate Na(OR₅), K(OR₅) wherein R₅ is C₁-C₆alkyl and a heterogeneous basic support selected from amberlite, ambersep, sepiolite, hydrotalcit or bentonit.

3. A process according to claim 1, wherein the copper salt is CuCl; the ligand is a phenanthroline ligand; the copper complex is [R₁R₂R₃R₄N](Cu₄OCl₁₀)] and the base is a quaternary ammonium salt or a hydrate thereof.

4. A process according to claim 1 wherein the catylytic system is CuCl/1,10-phenanthroline/[(CH₃)₄N]OH.5H₂O or CuCl/[(CH₃)₄N]OH.5H₂O.

5. A process according to claim 1, wherein the process is carried out in the presence of a solvent at a temperature in the range of 30-140° C.
